(19) United States
(12) Patent Application Publication
(10) Pub. No.: US 2002/0077609 A1
(43) Pub. Date: Jun. 20, 2002

(54) Disposable urinary collection bag cover

(76) Inventor: Madeline Johnson, Waco, TX (US)

Correspondence Address: DAVID G. HENRY, 700 Texas Center, P.O. Box 1470, Waco, TX, 76703-1470, US

(21) Appl. No.: 09/957,691

(22) Filed: Sep. 19, 2001

Related U.S. Application Data
(63) Continuation-in-part of application No. 09/111,465, filed on Jul. 7, 1998, now abandoned.

(51) Int. Cl. A61M 1/00

(52) U.S. Cl. 604/317; 604/322

(57) ABSTRACT

A disposable urine collection bag cover which is configured for very easy application and removal from a urinary collection bag, is fabricated from disposable, medical grade fabric, is easily secured in place, and provides very inconspicuous “camouflage” for the bag. The bag is designed with pleats along its length to accommodate bags of varying initial sizes, as well as to accommodate the growth in size as the bag fills during use.

### CITATION TO PRIOR APPLICATION

This is a continuation-in-part of U.S. application Ser. No. 09/111,465 from which priority is claimed.

### BACKGROUND OF THE INVENTION

1. Field of The Invention

The present invention relates to accessories for use in patient case, and in particular relating to urinary collection bags.

2. Background Information

Improvements in patient care with respect to privacy has been an increasing concern of late. The issues have reached a level such that it has been specifically discussed and examined by such organizations as the Joint Commission for Accreditation of Healthcare Organizations, (JCAHO). Patient privacy and comfort with respect to urinary collection bags is a sub-set of the overall issues discussed.

Due to various health problems, many hospital patients, as well as many outpatients, must use urinary catheters with drainage collection bags attached. The presence, and in particular, the visibility of urine collection devices often causes patients and family members and visitors alike to experience a sense of awkward embarrassment. In this society, we are taught virtually all our life to “take care of bodily functions” in private, and that exposing others to our bodily waste (and its odors) is to be avoided as a matter of common courtesy. The resulting sensibilities do not disappear as soon as one enters a hospital, or otherwise suffers a health event that requires artificial urine collection.

To address this issue, some have, on an ad hoc basis, fabricated covers for urine collection bags. However, no one has provided a cover that is easy to use, inexpensive to replace (as it becomes soiled) and inconspicuous. Ways to attache or secure such ad hoc covers (made of regular garment fabric) include elastic bands, clips, and even tape. The hap hazzard nature and appearance of such covers often draws more attention to, rather that direct attention away from the collection bag.

It would well-serve those who must use urinary collection bags to provide some device which inconspicuously conceals urinary collection bags from view of visitors, is easy to use, accommodates collection bags of varying sizes, and is inexpensive to purchase initially and to replace once the over becomes soiled.

### SUMMARY OF THE INVENTION

In view of the foregoing, it is an object of the present invention to provide an improved urinary collection bag cover.

It is another object of the present invention is to provide individuals who must use bladder catheters attached to urine drainage bags with privacy by providing the individual with a cover that easily contains any such bag.

Another object of the invention is to provide a cover that will fit snugly on to any size urine drainage bag and yet will easily enlarge as the bag fills with urine.

A still further object of the present invention is to provide a cover as described which easily adapts to receive empty urine collection bags, and deliver up filled urine collection bags, and in between such operations, remain secured to the bag and fully conceal its presence from view.

In satisfaction of these and related objects, the present invention provides a disposable urine collection bag cover which is configured for very easy application and removal from a urinary collection bag, is fabricated from disposable, medical grade fabric, is easily secured in place, and provides very inconspicuous “camouflage” for the bag. The bag is designed with pleats along its length to accommodate bags of varying initial sizes, as well as to accommodate the growth in size as the bag fills during use.

### BRIEF DESCRIPTION OF THE DRAWINGS

FIG. 1 is an elevational frontal view of the urinary collection bag cover of the present invention.

FIG. 2 is an elevational backside view of the cover of FIG. 1.

FIG. 3 is an exploded side view of the cover of FIGS. 1 and 2.

### DETAILED DESCRIPTION OF THE PREFERRED EMBODIMENT

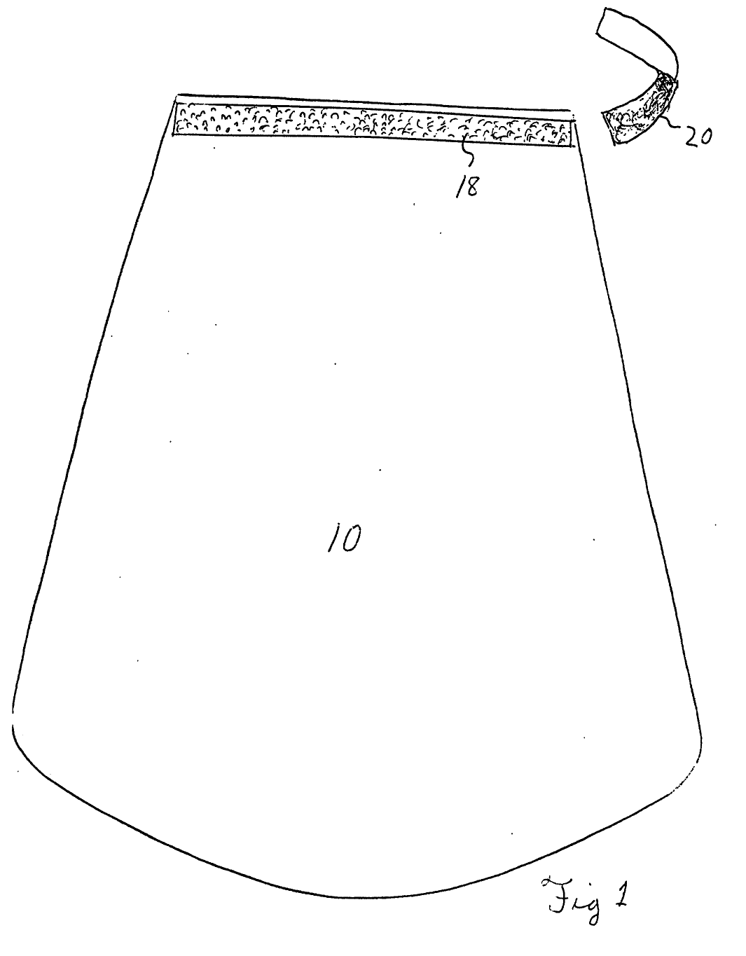

Referring to FIG. 1, the urinary collection bag cover of the present invention is identified generally by the reference numeral 10. Cover 10 is a bag-like structure that, according to the preferred mode of manufacture, is made from two sheets of medical grade disposable sheeting which is sewn together when “turned inside-out” and then reversed for use (much as a pillow case is made). In the alternative, other manufacturing methods may be apparent to those skilled in the art, including through sonic welding of suitable materials. The medical grade sheeting is, in the preferred embodiment, the same as is used for disposable surgery smocks, shoe covers, and the like as are used in surgery suites, and is well know to those in the medical supply field. Not only is such material suitable for medical use, buts its visual impact is so consistent with materials normally seen in the health care context, that it does not draw attention, as some prior art covers made from garment fabric tend to do.

Cover 10 of the present invention may be made of any size, but should be made to accommodate the largest of sizes of urinary collection bags in common use.

Precisely because urinary collection bags do vary in size, and expand during use, the present cover 10 is designed with deep pleats 12 on either of its two sides.

At a top margin of bag 10 is provided an orifice or opening 14 through which a urinary collection bag is inserted and removed. Pleats 12 extend to the edge of orifice 14, such that orifice 14 expands and contracts, respectively, to enlarge for admitting and delivering up the to-be-covered urinary collection bag and to secure the cover 10 to the urinary collection bag, while still allowing the passage of the necessary tube(s) that go to/from the collection bag.

To maximize ease of use, several securing schemes for securing cover 10 to a urinary collection bag and maintaining the greatest possible concealment of the bag were examined by the present inventor. One attachment system or method appears to be ideal, and experiments indicate that such provides a considerable advantage over approaches taken earlier by the present inventor, and by those who fabricated the earlier-mentioned ad hoc covers.

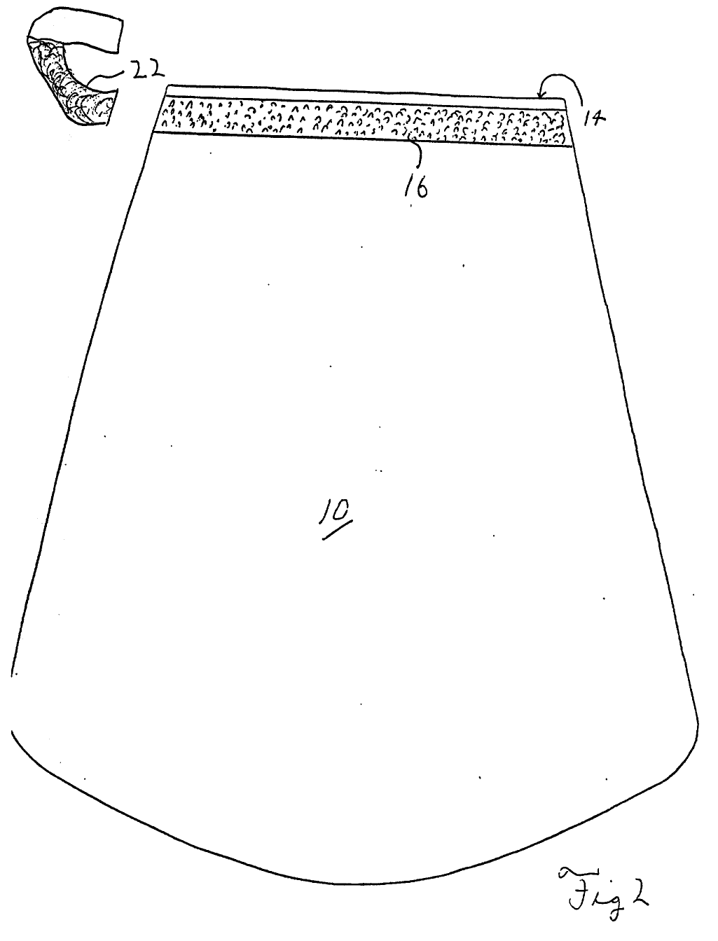

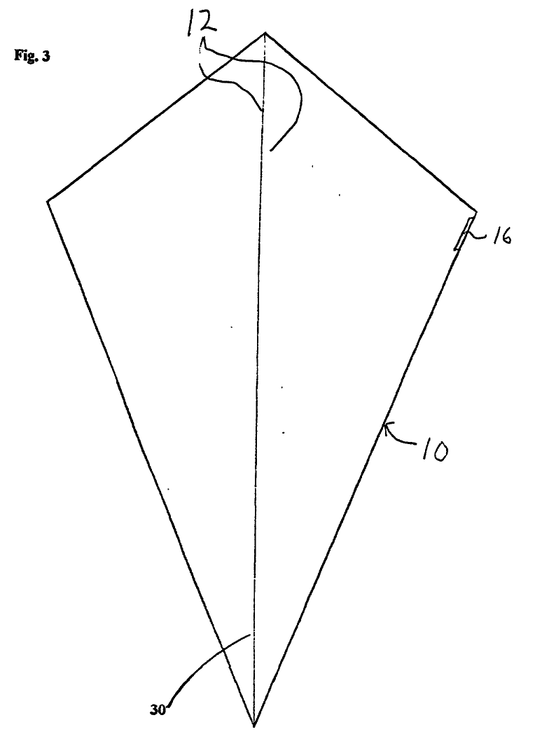

Referring again to FIGS. 1-3, adjacent to orifice 14 are attached to cover 10 two strips 16 and 18 of the hook material of conventional hook and loop fasting material (VELCRO). Strips 16 and 18 extend along lengths or the margin of bag 10 near orifice 14 between and extending substantially to pleats 12. Two strips 20 and 22 (loop material of the hook and loop paring) are provided to attach to and span the juxtaposed ends of strips 16 and 18 to secure orifice 14 in its contracted configuration as between insertions or removals of a urinary collection bag.

It should be noted that, with this arrangement, strips 20 and 22 (or either of them) may be removed from either of strips 16 or 18 at any given time, to allow maximum flexibility in working with cover 10. There is, as a result, no “front” or “back” of bag 10 for purposes of working with cover 10. A more conventional approach might have been to securely attach spanning strips to one side or other of the two pleats 14, but such creates a “front” and “back” for cover 10, and reduces is convenience.

The present invention will provide the advantage of being easy to use by both the patient and health care worker. It will increase patient privacy and decrease patient self consciousness. The simplicity of the invention also makes it easy to manufacture.

Although the invention has been described with reference to specific embodiments, this description is not meant to be construed in a limited sense. Various modifications of the disclosed embodiments, as well as alternative embodiments of the inventions will become apparent to persons skilled in the art upon the reference to the description of the invention. It is, therefore, contemplated that the appended claims will cover such modifications that fall within the scope of the invention.

I claim:

1. A disposable urinary collection bag privacy cover comprising:
a cover member constructed of fabric and sized and contoured for enveloping said urinary collection bag in a wrap-around configuration, said cover member being further configured for defining an orifice through which one or more tubes leading to and from said urinary collection bag may extend when said urinary collection bag cover is engaged with said urinary collection bag;
reversibly attachable and detachable connection means affixed to said cover member near margins of said cover member for facilitating alternating joinder and disengagement of said margins for, when joined, maintaining said cover member in said wrap-around configuration about said urinary collection bag and, when disengaged, removal of said cover member from said urinary collection bag.

2. The privacy cover of claim 1 wherein said cover member is fashioned from designated disposable fabric.

3. The privacy cover of claim 2 wherein said reversibly attachable and detachable connection means is fashioned from hook and eye material whereby hook batting is affixed to one of two margins of said cover member to be joined and an eye assembly is affixed to the second such margin.
